(19) United States
(12) Patent Application Publication
(10) Pub. No.: US 2024/0215810 A1
(43) Pub. Date: Jul. 4, 2024

(54) ENDOSCOPE VALVE DEVICES, SYSTEMS, AND METHODS

(71) Applicant: BOSTON SCIENTIFIC SCIMED, INC., MAPLE GROVE, MN (US)

(72) Inventor: Nathan Thomas Cummings, Worcester, MA (US)

(73) Assignee: BOSTON SCIENTIFIC SCIMED, INC., MAPLE GROVE, MN (US)

(21) Appl. No.: 18/402,070

(22) Filed: Jan. 2, 2024

Related U.S. Application Data
(60) Provisional application No. 63/436,758, filed on Jan. 3, 2023.

(51) Int. Cl. A61B 1/015; A61B 1/00

(52) U.S. Cl. CPC A61B 1/015; A61B 1/00039

(57) ABSTRACT

A valve shaft for a valve assembly of a medical device. The valve shaft is shiftable between an off position in which the valve assembly is in an off configuration and does not fluidly communicate a fluid source with a patient, and an on position in which the valve assembly is in an on configuration fluidly communicating a fluid source with a patient. The valve shaft is formed of a valve shaft proximal component formed of a first material, and a valve shaft distal component formed of a second material. The first material is more rigid than the second material so that the valve shaft proximal component may have hard stop features limiting axial and/or rotational movement of the valve shaft. The second material is capable of forming a seal between the valve shaft distal component and another component of the valve assembly.

### CROSS-REFERENCE TO RELATED APPLICATIONS

This application claims the benefit of priority under 35 U.S.C. § 119 to U.S. Provisional Application No. 63/436,758, filed Jan. 3, 2023, the entire disclosure of which is hereby incorporated by reference herein for all purposes.

### FIELD

The present disclosure relates generally to devices (including, without limitation, components and assemblies), systems, and methods for controlling flow of materials through a valve. In particular, the present disclosure relates to devices, systems, and methods for controlling flow of materials through a valve assembly usable in a medical device such as an endoscope.

### BACKGROUND

Various endoscopes are known in the art for use during various medical procedures. Endoscopes typically have an insertion tube with a working channel via which substances (e.g., fluids such as gas or liquids) or devices or instruments or tools may be introduced, or substances may be removed or suctioned out. Endoscopes thus typically also include control handles with various actuators, connections, etc., configured to control the endoscope (e.g., navigation of the endoscope), and/or materials, substances, devices, systems, instruments, tools, etc., delivered through the working channel. For instance, the actuators, connections, etc., may be engaged to supply fluid to an anatomical site (such as for irrigation) and/or to apply suction to the anatomical site (such as to withdraw materials therefrom) via the insertion tube. A fluid supply and/or vacuum source may be fluidly coupled to the endoscope's insertion tube via the endoscope's control handle. To control flow of substances through an endoscope, a fluid source, and/or a suction pump/vacuum source is fluidly coupled with the endoscope handle and the insertion tube via a valve assembly. The valve assembly typically has a valve well and a valve shaft shiftable within the valve well between an off position in which the valve assembly is in an off/closed configuration, and an on position in which the valve assembly is in an on/open configuration. In the off configuration, the valve assembly blocks fluid communication between the fluid source/suction source and the insertion tube of the endoscope. When the valve assembly is shifted into an on configuration (typically by being depressed with respect to the handle), fluid communication between the fluid source/suction source and the working channel of the endoscope is established to supply fluid and/or to apply suction/negative pressure to the insertion tube of the endoscope. Proper sealing of the ports, channels, lumens, etc., associated with such valve assemblies is important. However, seals which provide sealing interference can also create a large drag force which may affect operation of the valve shaft as it is shifted, often repeatedly, within the valve well. Moreover, if sealing material is overmolded over a portion of a valve shaft, such overmolding may result in a reduction in the cross-sectional dimensions of the valve shaft, reducing the inner diameter of a suction channel through the valve shaft. Moreover, a valve assembly of an endoscope is typically biased into an off configuration as the suction source continuously applies suction to the valve assembly. If the valve shaft is made of a single, compliant material, such material may experience creep/relaxation due to a pre-load force applied thereto in a packaged/resting state to maintain the suction valve assembly in a closed, off configuration. There remains a need for improvements to endoscope valves, such as suction valves and seals associated therewith.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a valve shaft is formed in accordance with various principles of the present disclosure for a valve assembly of a medical device. The valve shaft has a valve shaft proximal component formed of a first material, and a valve shaft distal component formed of a second material. The first material is more rigid than the second material; and the second material is configured to seal a port defined in a valve well of the valve assembly into which the valve shaft is to be extended. The valve shaft is shiftable within the valve well channel along an actuation axis and between an off position and an on position. When the valve shaft is in the off position, the valve assembly is in an off configuration. When the valve shaft is in the on position, the valve assembly is in an on configuration.

In some embodiments, the valve shaft distal component is formed of foam; and the valve shaft proximal component has a distal extension configured to extend into the valve shaft distal component. Optionally, the distal extension of the valve shaft proximal component comprises one or more barbs engaging within a proximal end of the valve shaft distal component to resist separation of the valve shaft proximal component from the valve shaft distal component.

Optionally, the valve shaft proximal component and the valve shaft distal component are secured together by being at least one of insert molded, overmolded, snap-fitted, interference fitted, welded, bonded, or adhered together.

Optionally, the valve shaft distal component has an outer diameter larger than an inner diameter of the valve well channel of the valve assembly in which said valve shaft is to be extended.

In some embodiments, the valve well includes a valve-well-suction-source port configured to be fluidly communicated with a suction source. When the valve shaft is in the off position, suction is not applied by the valve assembly; and when the valve shaft is in the on position, suction may be applied by the valve assembly.

In some embodiments, the valve shaft distal component defines a valve shaft suction channel therethrough extending along the actuation axis and in fluid communication with a valve-well-suction-source port; the valve shaft further defines a valve-shaft-suction-application port extending transverse to the actuation axis; when the valve shaft is in the off position, the valve shaft distal component seals a valve-well-suction-application port from fluid communication with the valve-well-suction-source port; and when the valve shaft is in the on position, the valve-shaft-suction-application port is in fluid communication with the valve-well-suction-application port to put the valve-well-suction-application port in fluid communication with the valve-well-suction-source port via the valve shaft suction channel. In some embodiments, the valve shaft suction channel and the valve shaft suction application port are defined in the valve shaft distal component distal to the distal end of the valve shaft proximal component. In some embodiments, the valve shaft distal component includes one or more circumferential seal elements extending circumferentially around and radially outwardly therefrom to seal with respect to the valve well channel. Optionally, the one or more circumferential seal elements are axially spaced apart from one another along the actuation axis. Optionally, the valve assembly defines bleed passages in fluid communication with the valve shaft suction channel and the valve-well-suction-source port when the valve shaft is in the off position, and sealed from fluid communication with the valve shaft suction channel and the valve-well-suction-source port by at least one of the circumferential seal elements when the valve shaft is in the on position.

Optionally, the valve assembly has s a valve cap coupled to the valve well; the valve shaft is axially shiftable with respect to the valve cap between the off position and on position; and the valve shaft proximal component comprises one or more hard stop features engaging the valve cap to limit axial and/or rotational movement of the valve shaft with respect to the valve cap. Optionally, the valve cap is rotationally fixed with respect to the valve well; and the valve shaft is rotationally fixed with respect to the valve cap and axially shiftable with respect thereto.

In accordance with various principles of the present disclosure, a valve shaft for a valve assembly is configured to be shifted between an off configuration and an on configuration by shifting the valve shaft between an off position and an on position, respectively, and the valve shaft includes a valve shaft proximal component formed of a first material; and a valve shaft distal component formed of a second material. The first material is more rigid than the second material; and the second material is formed of a sealing material capable of sealing a suction path or a bleed path through a valve assembly.

In some embodiments, the valve shaft distal component is formed of foam; and the valve shaft proximal component has a distal extension configured to extend into the valve shaft distal component. Optionally, the distal extension of the valve shaft proximal component comprises one or more barbs engaging within a proximal end of the valve shaft distal component to resist separation of the valve shaft proximal component from the valve shaft distal component.

Optionally, the valve shaft proximal component and the valve shaft distal component are secured together by being one of insert molded, overmolded, snap-fitted, interference fitted, welded, bonded, or adhered together.

In some aspects, an endoscope is formed accordance with various principles of the present disclosure with a control handle including a valve assembly; a connector cord configured to fluidly couple the control handle with a fluid source; and an insertion tube coupled to the control handle and configured to be fluidly coupled with the fluid source via the control handle. The valve assembly includes a valve shaft shiftable with respect to the control handle along an actuation axis and between an off position and an on position. When the valve shaft is in the off position, the valve shaft seals the insertion tube from fluid communication with the fluid source. When the valve shaft is in the on position, the insertion tube is in fluid communication with the fluid source via the valve assembly. The valve shaft has a valve shaft proximal component formed of a first material, and a valve shaft distal component formed of a second material. The second material is capable of forming a seal with one or more components of the valve assembly, and the first material is more rigid than the second material.

Optionally, the valve shaft proximal component comprises one or more hard stop features engaging another component of the valve assembly to limit axial and/or rotational movement of the valve shaft with respect to a port in a valve well of the valve assembly.

Optionally, the valve assembly defines bleed passages in fluid communication with the fluid source when the valve shaft is in the off position, and sealed by the valve shaft distal component from fluid communication with the fluid source when the valve shaft is in the on position.

In accordance with various principles of the present disclosure, a method of forming a valve shaft for valve assembly for a medical instrument includes forming a valve shaft proximal component of a first material; and forming a valve shaft distal component extending distally away the valve shaft proximal component and of a second material capable of forming a seal with one or more components of the valve assembly, and less rigid than the first material.

Optionally, the method further includes separately forming and then coupling together the valve shaft proximal component and the valve shaft distal component.

Optionally, the method further includes molding a proximal end of the valve shaft distal component over a distal end of the valve shaft proximal component and distally beyond the distal end of the valve shaft proximal component.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### BRIEF DESCRIPTION OF THE DRAWINGS

Non-limiting embodiments of the present disclosure are described by way of example with reference to the accompanying drawings, which are schematic and not intended to be drawn to seale. The accompanying drawings are provided for purposes of illustration only, and the dimensions, positions, order, and relative sizes reflected in the figures in the drawings may vary. For example, devices may be enlarged so that detail is discernable, but is intended to be sealed down in relation to, e.g., fit within a working channel of a delivery catheter or endoscope. In the figures, identical or nearly identical or equivalent elements are typically represented by the same reference characters, and similar elements are typically designated with similar reference numbers differing in increments of 100, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an example of an embodiment of an endoscope with one or more valves formed in accordance with aspects of the present disclosure.

FIG. 2 illustrates a perspective view of an example of an embodiment of a valve assembly formed in accordance with various principles of the present disclosure, such as for an endoscope such as illustrated in FIG. 1, in an off configuration.

FIG. 3 illustrates a perspective view of an example of an embodiment of a valve assembly formed in accordance with various principles of the present disclosure, such as for an endoscope such as illustrated in FIG. 1, in an on configuration.

FIG. 4 illustrates a perspective view of an example of an embodiment of a valve shaft formed in accordance with various principles of the present disclosure for a valve assembly such as illustrated in FIG. 2 and FIG. 3.

FIG. 5 illustrates a perspective view of an example of an embodiment of a valve shaft formed in accordance with various principles of the present disclosure for a valve assembly such as illustrated in FIG. 2 and FIG. 3.

FIG. 6A illustrates a cross-sectional view, such as along line IIA-IIA of a valve assembly such as illustrated in FIG. 2, of an example of an embodiment of a valve assembly in an off or closed configuration.

FIG. 6B illustrates a cross-sectional view, such as along line IIB-IIB of a valve assembly such as illustrated in FIG. 3, of an example of an embodiment of a valve assembly in an on or open configuration.

FIG. 7A illustrates a cross-sectional view, such as along line IIA-IIA of a valve assembly such as illustrated in FIG. 2, of an example of an embodiment of a valve assembly in an off or closed configuration.

FIG. 7B illustrates a cross-sectional view, such as along line IIB-IIB of a valve assembly such as illustrated in FIG. 3, of an example of an embodiment of a valve assembly in an on or open configuration.

FIG. 8A is a bottom perspective view of a cap portion of a valve assembly as illustrated in FIG. 2 or FIG. 3.

FIG. 8B is a top perspective view of a valve well of a valve assembly as illustrated in FIG. 2 or FIG. 3.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. As used herein, a “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location.

Various medical devices include valve assemblies to regulate or control fluid delivery (irrigation) or fluid suction (aspiration) with respect to an anatomical site. Although the present disclosure describes suction valves, it will be appreciated that the principles of the present disclosure need not be so limited.

A suction valve assembly of a medical device is arranged to apply suction from a suction source to an anatomical site via a flexible tubular element which is configured and positionable with respect to the anatomical site. The medical device may be an endoscope, and the flexible tubular element may be an insertion tube of the endoscope. The suction source may be a pump or other mechanism creating a vacuum to be applied to the anatomical site via the flexible tubular element. In the off configuration of the valve assembly, fluid communication between the suction source and the flexible tubular element is cut off or blocked so that suction is not applied to the anatomical site, and the valve may be considered to be in a closed configuration. In the on configuration of the valve assembly, the suction source is fluidly coupled with the flexible tubular element, such as to aspirate an anatomical site, and the valve may be considered to be in an open configuration.

Valve assemblies of medical devices may be mounted with respect to a control handle, and typically include an actuatable member movable with respect to the control handle to shift the valve assembly between the off configuration and the on configuration. The actuatable member may include a valve shaft and a user-engagement element which are movable with respect to a valve well formed in, or formed and positioned within, the control handle. Holes may be formed in the valve shaft to form channels which may be selectively aligned with ports in the valve well to selectively place the suction source in and out of fluid communication with the flexible tubular element of the device to apply suction to an anatomical site or to not apply suction. Various control handles have different arrangements of ports and flow paths placing the suction source in and out of fluid communication with the flexible tubular element to be directed to the anatomical site. Accordingly, various arrangements of holes within valve shafts may place ports to suction sources and ports to a flexible tubular element (directed to a patient) in and out of alignment with each other.

For the sake of convenience, and without intent to limit, reference is made herein to a valve assembly for a suction valve of an endoscope. The flexible tubular element of the endoscope is referenced herein as an insertion tube, and is generally positionable within a patient, such as within an organ, body lumen/passageway, cavity, etc. (reference being made herein to any or other such anatomical sites without intent to limit). The insertion tube defines one or more lumens therethrough configured for passage of materials, instruments, tools, devices, etc., through the working channel to an anatomical site. For instance, the lumens may include a suction lumen, an irrigation lumen, a working channel, and a visualization lumen (e.g., for a light guide, optic fiber, camera element, etc.). The present disclosure describes valve assemblies usable in a control handle with a suction-source port fluidly communicating a suction source with a suction-source port of a valve well, a suction source port of a valve shaft, and an axially-extending suction channel through the valve shaft. More particularly, the axially-extending suction channel extends generally parallel to the direction of actuation movement of the actuatable member of the valve assembly, and thus generally parallel to the longitudinal axis of the valve shaft. The valve shaft has a suction-application port extending transverse to the direction of actuation movement of the valve shaft. The suction-application port of the valve shaft is selectively moved in and out of fluid communication with a suction-application port in the valve well of the valve assembly in fluid communication with a suction application port in the control handle to selectively apply suction to an anatomical site when the endoscope is in use. More particularly, when the valve assembly is in an off configuration, the valve shaft is in an off position with the valve shaft blocking fluid communication to the suction-application ports. When the valve assembly is in the on configuration, the valve shaft places the suction-application ports in fluid communication with the suction channel through the valve shaft and the suction-source ports. The suction application ports are in fluid communication with a suction application device, such as the suction lumen or working channel of an insertion tube of an endoscope, to apply suction to an anatomical site. Typically, the actuatable member of the valve assembly is biased into the off configuration, such as with a biasing element, so that suction is only applied when the medical professional intends to apply suction, such as by pressing on the actuatable member.

As may be appreciated, a tight tolerance fit between the valve shaft and the valve well is generally needed to create and maintain a good seal around the ports thereof. In accordance with various principles of the present disclosure, the valve shaft is formed a proximal component and a distal component connected together generally axially. The proximal component of the valve shaft is configured for actuation by a user of the valve assembly, and typically has a user-engagement element. Additionally, a biasing force is applied to the proximal component of the valve shaft to hold the valve shaft in the desired position, typically the off position. The distal component of the valve shaft is shifted within the valve well of the valve assembly between an off position closing/sealing the valve-well-suction-application port, and an on position in which the valve-well-suction-application port is open/in fluid communication with the valve-shaft-suction-application port and the suction-application channel through the valve shaft to allow suction from the suction-source ports to be applied to the suction-application ports. In accordance with various principles of the present disclosure, the distal component of the valve shaft is formed from a sealing material such as a gasket material (e.g., foam, rubber, etc.) capable of forming a seal with the valve-well-suction-application port. Moreover, in accordance with various principles of the present disclosure, the proximal component of the valve shaft is formed from a material sufficiently rigid to resist creep or other deformation which may otherwise be caused by the biasing force applied thereto. The proximal component is thus more rigid than the distal component. The proximal component and the distal component of the valve shaft may be formed separately and joined together, such as by mechanical interference fit or bonding. The proximal component and the distal component of the valve shaft may be molded together, such as insert molded.

Various embodiments of valve devices (including, without limitation, components and assemblies), systems, and methods will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

In the drawings, it will be appreciated that common features are identified by common reference elements and, for the sake of brevity and convenience, and without intent to limit, the descriptions of the common features are generally not repeated. For purposes of clarity, not all components having the same reference number are numbered. It will be appreciated that, in the following description, elements or components similar among the various illustrated embodiments are generally designated with the same reference numbers differing by a multiple of 100, and redundant description is generally omitted for the sake of brevity. Moreover, certain features in one embodiment may be used across different embodiments and are not necessarily individually labeled when appearing in different embodiments.

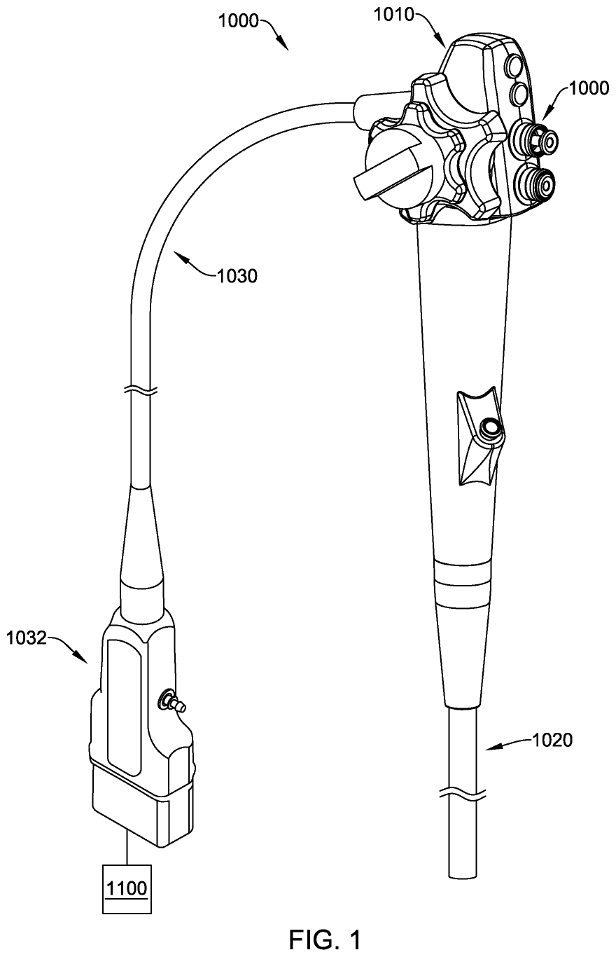

Turning now to the drawings, an example of an embodiment of a valve assembly 100 formed in accordance with various principles of the present disclosure is illustrated in FIG. 1 as provided in an example of an embodiment of an endoscope 1000. It will be appreciated that the endoscope 1000 is an example of an embodiment in which principles of the present disclosure may be applied, and that various principles of the present disclosure are applicable to other medical instruments to control fluid flow with respect thereto, the details of which are not critical to the present disclosure. Moreover, although reference is made to a suction valve, it will be appreciated that the disclosed principles and embodiments are applicable to other valves, such as fluid supply/irrigation valves.

The illustrated example of an embodiment of a valve assembly 100 is mounted with respect to a control handle 1010 of the endoscope 1000 to regulate the flow of materials (e.g., fluid) between the insertion tube 1020 of the endoscope 1000 and a suction source 1100. The endoscope 1000 has a connector cord 1030 extending to a scope connector 1032 with which the endoscope 1000 (and valve assembly 100) may be fluidly coupled with the suction source 1100. The connector cord 1030 may be alternatively referenced herein as an umbilical cord, umbilicus, universal cord, etc., without intent to limit. The scope connector 1032 may also couple the endoscope 1000, via the connector cord 1030, with a variety of components, devices, etc., such as a fluid source (to supply air, carbon dioxide, water, saline, or other gases or liquids), electrical connections, light sources, visualization elements (e.g., optic fibers, cameras, etc.), or other components, devices, etc., usable with the endoscope 1000. The insertion tube 1020 has a fluid lumen extending therethrough to a distal end which is positionable (insertable, navigable, etc.) with respect to an anatomical site (e.g., within a patient). Similarly, the connector cord 1030 has a fluid lumen extending therethrough to fluidly couple the suction source 1100 (e.g., via the scope connector 1032) with the control handle 1010. The fluid lumens through the insertion tube 1020 and the connector cord 1030, and the distal end of the insertion tube 1020, may be well-known features formed in a manner known to those of ordinary skill in the art and are not illustrated to simplify the drawings by eliminating details in the illustration of the endoscope 1000 in FIG. 1 which are not necessary for understanding the present disclosure.

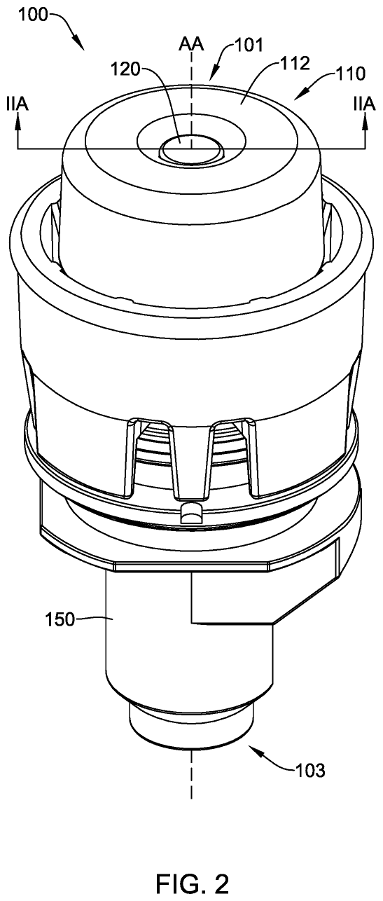

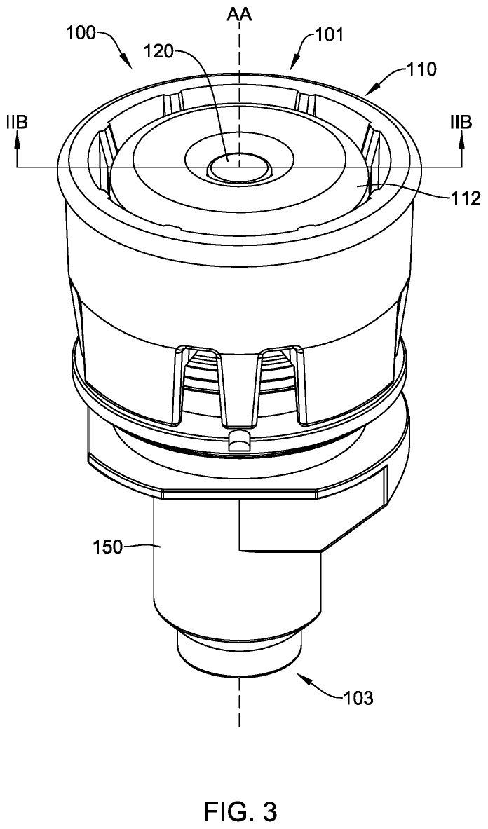

An example of an embodiment of a valve assembly 100 formed in accordance with various principles of the present disclosure is illustrated in FIG. 2 and FIG. 3, in isolation from an endoscope (such as the endoscope 1000 illustrated in FIG. 1). In the illustrated example of an embodiment, the valve assembly 100 includes an actuatable member 110 having a valve shaft 120 movable with respect to a valve well 150 of the valve assembly 100. Shifting of the actuatable member 110 and the valve shaft 120 between an off position (as illustrated in FIG. 2) and an on position (as illustrated in FIG. 3) shifts the valve assembly 100 respectively between an off configuration, in which the valve assembly 100 does not apply suction to a suction application device, and an on configuration, in which the valve assembly 100 may apply suction to a suction application device, as described in further detail below.

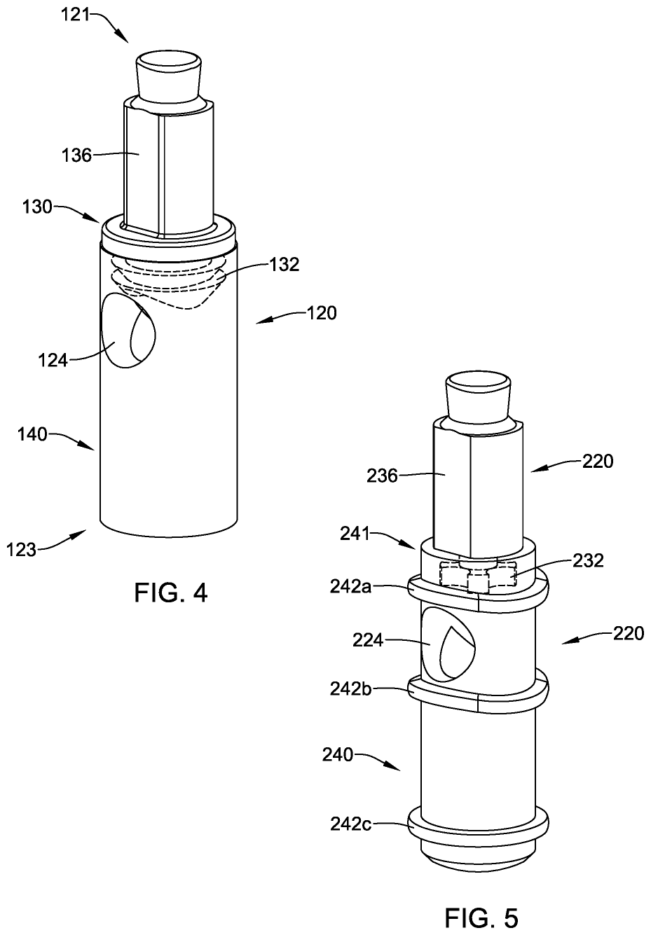

In accordance with various principles of the present disclosure, the valve shaft 120 is formed with a proximal component 130 and a distal component 140, such as illustrated in FIG. 4. An alternate embodiment of a valve shaft 220 formed with a proximal component 230 and a distal component 240, is illustrated in FIG. 5. Such configuration of the valve shafts 120, 220 allows for improved sealing capabilities, while also proving a durable component with reliably alignable valve ports, which is also resistant to deformation, as described in further detail below. It will be appreciated that the valve shafts 120, 220 illustrated in FIG. 4 and FIG. 5 can be arranged and operate in similar valve assemblies in substantially the same or similar manners. Accordingly, common elements of the valve shafts 120, 220, such as with common functions, are indicated with the same reference characters differing in value by 100. In general, references herein to and descriptions herein of one of the valve shafts 120, 2204 are applicable to the other of the valve shafts 120, 220 unless otherwise explicitly indicated., For the sake of simplicity and brevity, and without intent to limit, reference may be made to just the valve shaft 120, it being understood that, unless explicitly indicated, such descriptions are applicable to the valve shaft 220 as well. For the sake of convenience, and without intent to limit.

The valve shaft proximal component 130, 230 is coupled with a user-engagement element 112 (e.g., a cap or button) of the actuatable member 110 configured for engagement by a user to shift the actuatable member 110 and valve shaft 120, 220 along an actuation axis AA and between off and on positions. The user-engagement element 112 may be separately formed from and coupled to a proximal end 121, 221 of the valve shaft 120, 220 (e.g., in any of a variety of manners known to those of ordinary skill in the art), or, instead, may be integrally formed with the valve shaft 120, 220. The valve shaft distal component 140, 240 extends within the valve well 150 and is formed of a material capable of sealing suction paths through the valve assembly 100 in the on and off configurations of the valve assembly 100, as described in further detail below.

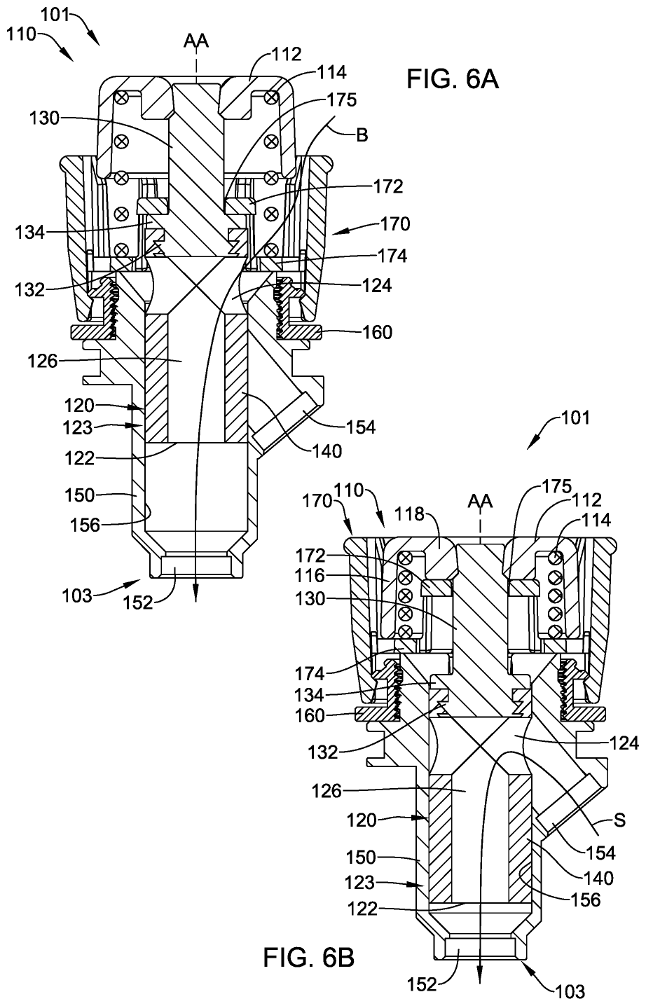

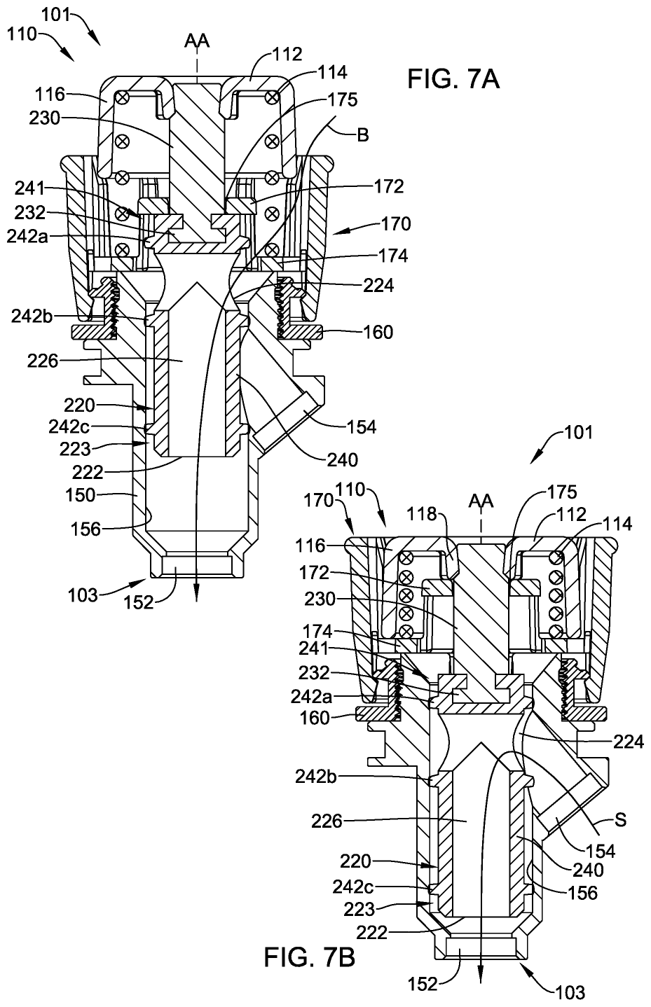

The positions of components 130, 140 and 230, 240 of the examples of embodiments of valve shafts 120, 220, respectively, relative to suction-source ports and suction-application ports of an example of an embodiment of a valve assembly 100 are illustrated in the cross-sectional views of FIG. 6A, FIG. 6B, FIG. 7A, and FIG. 7B. In particular, examples of embodiments of off and on configurations of an example of an embodiment of a valve assembly 100, and corresponding off and on positions of an actuatable member 110 and associated valve shaft 120, 220 thereof with respect to a valve well 150, are illustrated in FIG. 6A, FIG. 6B, FIG. 7A, and FIG. 7B.

As may be appreciated with reference to FIG. 6A, FIG. 6B, FIG. 7A, and FIG. 7B, the illustrated example of an embodiment of a valve well 150 of the valve assembly 100 has a valve-well-suction-source port 152 configured to be fluidly coupled with a suction source (such as a suction source 1100 as illustrated in FIG. 1), and a valve-well-suction-application port 154 configured to be fluidly coupled with a suction application device (such as an insertion tube 1020 as illustrated in FIG. 1). The valve-well-suction-source port 152 extends generally along the actuation axis AA of the valve assembly 100, whereas the valve-well-suction-application port 154 extends transverse to the actuation axis AA (and thus may be considered a side port). The illustrated example of an embodiment of a valve well 150 is formed separately from and inserted into the control handle 1010 of an endoscope 1000 such as illustrated in FIG. 1, although the present disclosure is not limited in this regard. A valve well nut 160 may hold the valve well 150 in place with respect to a control handle (such as the control handle 1010 illustrated in FIG. 1), such as in a manner known to those of ordinary skill in the art. An example of an embodiment of a cap 170 is illustrated as coupled with respect to the valve well 150 via the valve well nut 160, however the present disclosure is not limited to the illustrated arrangement. The valve shaft 120, 220 extends through a shaft-receiving through-hole 175 defined through a radially-inwardly-extending limit shoulder 172 within the cap 170. The cap 170 may provide various features for assembly and use of the valve assembly 100, such as guides for actuation movement of the actuatable member 110, orientation features for the actuatable member 110, suction bleed passages through the valve assembly 100, and other features, as described in further detail below. In the illustrated example of an embodiment, the cap 170 is a single piece element, however, the cap 170 may alternatively be formed as a two-piece element without impacting the present disclosure.

The examples of embodiments of valve shafts 120, 220 illustrated in FIG. 6A, FIG. 6B, FIG. 7A, and FIG. 7B have a valve-shaft-suction-source port 122, 222 defined at the distal end 123, 223 of the valve shaft 120, 220. The valve-shaft-suction-source port 122, 222 is in fluid communication with a valve shaft suction channel 126, 226 extending generally axially through the valve shaft 120, 220 along the longitudinal axis LA of the valve shaft 120, 220. As may be appreciated, the valve-shaft-suction-source port 122, 222 remains in fluid communication with the valve-well-suction-source port 152 while the valve shaft 120, 220 is in the off position as well as the on position. The valve shaft 120, 220 also includes a valve-shaft-suction-application port 124, 224 extending transverse to the longitudinal axis LA of the valve shaft 120, 220 (and thus may be considered a side port), and in fluid communication with the valve shaft suction channel 126, 226.

The actuatable member 110, with its associated valve shaft 120, 220, is shiftable along the actuation axis AA to shift the valve-shaft-suction-application port 124, 224 into and out of fluid communication with the transversely-extending valve-well-suction-application port 154. When the actuatable member 110 is in the off position, the valve shaft 120 is in an off position and the valve assembly 100 is in an off configuration with the valve-shaft-suction-application port 124 out of alignment and not in fluid communication with the valve-well-suction-application port 154. Accordingly, the valve shaft suction channel 126, 226 (and the suction source fluidly coupled therewith) is not in fluid communication with the valve-well-suction-application port 154. As such, the valve assembly 100 is in an off configuration and does not apply suction. When the actuatable member 110 is actuated to move the valve assembly 100 into an on configuration, the valve shaft 120, 220 is shifted into an on position to align the valve-shaft-suction-application port 124, 224 with the valve-well-suction-application port 154. The valve-well-suction-application port 154 is thereby placed in fluid communication with the valve-shaft-suction-application port 124, 224 and thus in fluid communication with the valve shaft suction channel 126, 226, and the valve-well-suction-source port 152 and the suction source. As such, the valve assembly 100 is in an on configuration and is capable of applying suction along a suction path S.

Typically, when a valve assembly 100 such as described herein is configured for use with an endoscope 1000, the suction source coupled to the valve assembly 100 is continuously running. However, it is generally desirable to limit suction applied by the valve assembly 100 to instances when suction is desired, and to limit, and preferably eliminate, suction force to the valve-well-suction-application port 154 when suction is not desired. For instance, in certain endoscopic procedures, it is desirable to maintain an anatomical site insufflated to improve visualization of the target site of the procedure, and/or to irrigate a target site, such as by supplying fluid to the target site. Suction may be limited to reducing the supplied fluid in certain instances, and/or to remove other materials (e.g., biological materials) from the target site. In such instances, the neutral position of the actuatable member 110 and valve shaft 120, 220 is typically an off position. In the examples of embodiments illustrated in FIG. 6A, FIG. 6B, FIG. 7A, and FIG. 7B, a biasing element 114 is provided to bias the actuatable member 110 and valve shaft 120, 220 into such position. The biasing element 114 may be a coil spring or other element capable of holding elements apart yet allowing such elements to be selectively moved together upon application of force to at least one of the elements and/or to the biasing element. In the examples of embodiments illustrated in FIG. 6A, FIG. 6B, FIG. 7A, and FIG. 7B, the biasing element 114 may be positioned between an underside of the user-engagement element 112 and a radially-inwardly-extending spring support 174 of the cap 170 to bias the actuatable member 110 into a neutral, off configuration (with the user-engagement element 112 biased proximally away from the cap 170 and valve well 150), such as in a manner known by those of ordinary skill in the art. In the illustrated examples of embodiments, the actuatable member 110 of the valve assembly 100 is shifted distally from a neutral position, in which the valve assembly 100 is in an off configuration, to shift the valve assembly 100 into on configuration. For instance, the user-engagement element 112 may be shifted from a position proximal to the proximal end 101 of the valve assembly 100 distally toward the distal end 103 of the valve assembly 100. However, principles of the present disclosure may be applied to other arrangements as well.

In accordance with various principles of the present disclosure, the valve shaft 120, 220 has a valve shaft distal component 140, 240 which is formed of a material which provides sealing of the valve-well-suction-application port 154 with respect to the valve shaft suction channel 126, 226 of the valve shaft 120, 220 when the actuatable member 110 and valve shaft 120, 220 are in the off configuration as illustrated in FIG. 6A, FIG. 7A. As such, a valve shaft 120, 220 formed in accordance with various principles of the present disclosure may provide improved sealing of the valve-well-suction-application port 154 when the valve assembly 100 is in an off configuration.

In the example of an embodiment of a valve shaft 120 illustrated in FIG. 4, the valve shaft distal component 140 is formed from a compressible material capable of forming a seal with and around the valve-well-suction-application port 154 when the valve shaft 120 is in an off position (such as illustrated in FIG. 6A). The seal created by the material of the valve shaft distal component 140 should be capable of cutting off suction to the valve-well-suction-application port 154 when the valve assembly 100 is in an off configuration. The valve shaft distal component 140 may be formed from a compressible, closed-cell foam (e.g., closed-cell plastic or rubber foam material) to achieve such sealing. Moreover, the outer diameter of the valve shaft distal component 140 may be oversized with respect to the inner diameter of the valve-well channel 156 defined in the valve well 150 and through which the valve shaft 120 axially shifts between the valve shaft 120 off and on positions. Such relative diameters of the valve shaft distal component 140 and the valve-well channel 156 balance the competing demands of sealing and shaft movement, and allow the valve shaft distal component 140 to move with respect to as well as to push against the valve-well channel 156 to create the desired seal with respect to the valve-well-suction-application port 154. Material selection may also facilitate the desired sealing and movability of the valve shaft distal component 140 with respect to the valve-well channel 156, a plastic foam typically providing the desired characteristics. Additionally, the increased diameter of the valve shaft distal component 140 compensates for negative pressure within the valve shaft suction channel 126 applied by the suction source (via the valve-well-suction-source port 152 and the valve-shaft-suction-source port 122), and resists collapse of the valve shaft distal component 140 which otherwise may occur from the negative pressure therein. The valve shaft suction channel 126 may be produced by extrusion during manufacture of the valve shaft distal component 140, and/or may be cut from a solid cylinder of material from which the valve shaft distal component 140 is formed. It will be appreciated that a valve shaft suction channel 126 formed by extrusion may provide a smoother wall, producing less turbulence, and reducing the likelihood of suctioned materials from becoming trapped within the wall of the valve shaft distal component 140. The valve-shaft-suction-application port 124 may be formed by die-cutting the valve shaft distal component 140, such as transverse to the valve shaft suction channel 126. The exterior surface of the valve shaft distal component 140 may have a relatively smooth surface to produce low drag forces as the valve shaft distal component 140 shifts between its off and on positions (which may occur multiple times during use of the valve assembly 100).

The valve shaft proximal component 130 may include a distal extension 132 configured to engage the valve shaft distal component 140 firmly to secure the components 130, 140 together to form the valve shaft 120. For instance, as may be appreciated with reference to FIG. 6A and FIG. 6B, and as illustrated in phantom in FIG. 2, the distal extension 132 may include one or more barbs fitted within the valve shaft distal component 140, such as within the valve shaft suction channel 126. The distal extension 132 is sized, shaped, configured, and/or dimensioned to engage the valve shaft distal component 140 to resist separation therefrom, such as may appreciated by those of ordinary skill in the art. As may be appreciated, the configuration of the distal extension 132 which engages the valve shaft distal component 140 need not extend continuously around the circumference of the valve shaft proximal component 130. For instance, discrete barbs arrayed around the circumference of the valve shaft proximal component 130 may be sufficient to resist relative movement of the valve shaft proximal component 130 and the valve shaft distal component 140.

In the example of an embodiment of a valve shaft 220 illustrated in FIG. 5, the valve shaft distal component 240 is formed from a compliant material capable of forming a seal with and around the valve-well-suction-application port 154 when the valve shaft 220 is in an off position (such as illustrated in FIG. 7A). The seal created by the material of the valve shaft distal component 240 should be capable of cutting off suction to the valve-well-suction-application port 154 when the valve assembly 100 is in an off configuration. In some embodiments, the valve shaft distal component 240 includes one or more circumferential seal elements 242a, 242b, 242c extending circumferentially around and radially outwardly from the valve shaft distal component 240. The circumferential seal elements 242a, 242b, 242c may be positioned with respect to the valve-well-suction-application port 154 to maintain appropriate sealing with respect thereto. For instance, when the valve shaft 220 is in the off configuration, as illustrated in FIG. 7A, the intermediate circumferential seal element 242b creates a seal proximal to the valve-well-suction-application port 154, and the distal circumferential seal element 242c creates a seal distal to the valve-well-suction-application port 154, thereby securely sealing the valve-well-suction-application port 154 from the suction source. When the valve shaft 220 is in the on configuration, as illustrated in FIG. 7B, the intermediate circumferential seal element 242b may create a seal distal to the valve-well-suction-application port 154, and the proximal circumferential seal element 242c may create a seal proximal to the valve-well-suction-application port 154, thereby securely sealing the valve-well-suction-application port 154 with respect to the valve shaft suction channel 126 and the suction source, thus eliminating leakage of suction The valve shaft distal component 240 may be formed from materials such as, without limitation, rubber, a thermoplastic elastomer (“TPE”), silicone, etc.

The separately formed valve shaft distal component 240 may be insert molded, overmolded, snap-fitted, interference fitted, welded, bonded, adhered, or otherwise secured (mechanically and/or chemically, in any acceptable manner known to those of ordinary skill in the art) to the valve shaft proximal component 230. The coupling of the valve shaft distal component 240 to the valve shaft proximal component 230 should be sufficiently secure so that the components 240, 230 do not rotate with respect to each other, thereby allowing repeatable, and accurate alignment of the valve-shaft-suction-application port 224 in the valve shaft distal component 240 with the valve-well-suction-application port 154. For instance, the valve shaft proximal component 230 may include a distal extension 232 (illustrated in phantom in FIG. 5) which is inserted into the proximal end 241 of the valve shaft distal component 240, or over which the proximal end 241 of the valve shaft distal component 240 may be formed (e.g., insert molded over). The distal extension 232 of the valve shaft proximal component 230 may be shaped (scalloped, knurled, or otherwise shaped with at least a noncircular region) to provide a mechanical interlock with the valve shaft distal component 240 to prevent relative rotation between the valve shaft proximal component 230 and the valve shaft distal component 240, especially during use.

Advantageously, as noted above, the valve shaft proximal components 130, 230 of the respective valve shafts 120, 220 are formed from a different material than the material of the valve shaft distal components 140, 240. In some aspects, the material of the valve shaft proximal components 130, 230 may be a relatively inflexible material which does not flex in a discernible manner during normal use or even during a resting state, in comparison with the more compliant sealing material of the valve shaft distal components 140, 240. The material of the valve shaft proximal component 130, 230 may be selected to withstand various axial forces impacting the valve shaft 120, 220, such as during use and/or in a packaged/resting state (e.g., a pre-load force applied thereto by the biasing element 114). In addition, formation of the valve shaft proximal component 130, 230 from a material which is more rigid than the material of the valve shaft distal component 140, 240) allows the valve shaft proximal component 130, 230 to withstand various hard stops for the movement of the actuatable member 110 and/or valve shafts 120, 220 to maintain alignment of the valve-shaft-suction-application port 124 and the valve-well-suction-application port 154. Additionally or alternatively, the material of the valve shaft proximal component 130, 230 may be selected to resist creep which may occur over time under the constant load of the biasing element 114 (such as over the course of a shelf life which may be two or even more years).

As discussed above, the actuatable member 110, and thus the valve shaft 120, 220, may be biased into a neutral position by a biasing element 114 positioned between the user-engagement element 112 of the actuatable member 110 and a radially-inwardly-extending spring support 174 of the cap 170. Because the user-engagement element 112 is coupled to a proximal end 121, 221 of the valve shaft 120, 220, the biasing force of the biasing element 114 also applies a biasing force to the valve shaft 120, 220. The biasing element 114 typically is a pre-loaded compression spring, exerting a continuous and constant biasing force on the actuatable member 110 while the device with the valve assembly 100 is packaged (before use), and in a neutral, resting position even during use. For instance, the biasing element 114 may have a preload of approximately 5 Newtons, and the shelf life of the device with the valve assembly 100 may be approximately two (2) years. Such continuous and constant force may cause a valve shaft formed of a material less rigid than the material selected for the valve shaft 120, 220 to stretch or otherwise exhibit creep, which may cause misalignment of the valve-shaft-suction-application port 124 and the valve-well-suction-application port 154 when the valve shaft 120, 220 is shifted from the neutral off position to the on position. As may be appreciated by those of ordinary skill in the art, formation of the valve shaft proximal component 130, 230 of a material more rigid than the material of the valve shaft distal component 140, 240 imparts the valve shaft proximal component 130, 230 with a greater ability to resist deformation than afforded by the valve shaft distal component 140, 240, while providing a valve shaft distal component 140, 240 with greater sealing capability than may be provided by the material of the valve shaft proximal component 130, 230. The valve shaft proximal component 130, 230 may be formed from a rigid plastic (e.g., acrylonitrile butadiene styrenes (ABS), polycarbonates (PC), blends thereof, etc.) or a metal, in contrast with the compliant material of the valve shaft distal component 140, 240.

As may be appreciated, in order to maintain a secure seal between the valve-shaft-suction-application port 124 and the valve-well-suction-application port 154, not just the sealing capabilities of the engaging materials, and not just the resistance to deformation/stretching, but also the axial and rotational alignment of the ports 124, 154 are important, and must be tightly controlled. In accordance with various principles of the present disclosure, formation of the valve shaft proximal component 130, 230 from a material more rigid than the material of the valve shaft distal component 140, 240 allows the formation of structures which allow for tight control of hard stops for axial travel of the valve shaft 120, 220 along the actuation axis AA as well as rotation of the valve shafts 120, 220 about the actuation axis AA.

Various features and/or structures may be provided on the actuatable member 110 to provide hard stops limiting axial movement of the actuatable member 110, and thus axial movement of the valve shaft 120, 220, to maintain accurate axial alignment of the valve-shaft-suction-application port 124, 224 and the valve-well-suction-application port 154. For instance, the actuatable member 110 may include one or more hard stop features interacting with one or more corresponding hard stop features on the cap 170. In the example of an embodiment of a cap 170 illustrated in FIG. 6A, FIG. 6B, FIG. 7A, and FIG. 7B, the radially-inwardly-extending limit shoulder 172 of the cap 170 not only defines the shaft-receiving through-hole 175 through which the valve shaft 120 extends, but also forms a proximal limit stop for the proximal travel of the valve shaft 120, 220. In the example of an embodiment of a valve shaft 120 illustrated in FIG. 4, the valve shaft proximal component 130 has a valve shaft proximal limit stop 134 in the form of a radially-outwardly extending flange or shoulder. As may be appreciated with reference to FIG. 6A, the proximal surface of the valve shaft proximal limit stop 134 abuts the distal surface of a cap proximal limit stop formed by the radially-inwardly-extending limit shoulder 172 of the cap 170 defining the shaft-receiving through-hole 175 through which the valve shaft 120 extends. As may be appreciated, formation of the valve shaft proximal component 130 from a material more rigid than the material of the valve shaft distal component 140 allows the valve shaft 120 to withstand biasing forces of the biasing element 114 pressing the valve shaft proximal limit stop 134 against the radially-inwardly-extending limit shoulder 172 to hold the valve shaft 120 in the off position. In the example of an embodiment of a valve shaft 220 illustrated in FIG. 5, the distal extension 232 of the valve shaft proximal component 230 imparts rigidity to the valve shaft distal component 140 which abuts the distal surface of the radially-inwardly-extending limit shoulder 172 of the cap 170, such as illustrated in FIG. 7A. Such interaction provides a hard stop to the proximal travel of the valve shaft 220 stronger than would be achieved by a valve shaft 220 formed completely of the material from which the valve shaft distal component 240 is formed.

In the example of an embodiment of a cap 170 illustrated in FIG. 6A, FIG. 6B, FIG. 7A, and FIG. 7B, the radially-inwardly-extending spring support 174 of the cap 170 not only provides a distal support for the biasing element 114, but also forms a distal limit stop for the proximal travel of the actuatable member 110 and thus the valve shaft 120, 220. For instance, a distal surface of the user-engagement element 112 may abut against the proximal surface of the radially-inwardly-extending spring support 174. In the example of an embodiment of an actuatable member 110 illustrated in FIG. 6B and FIG. 7B, the user-engagement element 112 has a circumferential skirt 116 which may be extended distally into engagement with the radially-inwardly-extending spring support 174 to form a hard stop for distal axial movement of the actuatable member 110. Additionally or alternatively, the user-engagement element 112 may have a cylindrical neck 118 extending around and engaging the proximal end 121, 221 of the valve shaft 120, 220. A distal end of the cylindrical neck 118 may engage the radially-inwardly-extending limit shoulder 172 of the cap 170 as a hard stop for distal axial movement of the valve shaft 120 through the shaft-receiving through-hole 175 in the radially-inwardly-extending limit shoulder 172. Additionally or alternatively, distal movement of the valve shaft 120, 220 may be limited by the solid height of the biasing element 114 (when the biasing element 114 bottoms out), and thus may contribute to ensuring axial alignment of the valve-shaft-suction-source port 122, 222 with the valve-well-suction-source port 152. As may be appreciated, formation of the valve shaft proximal component 130, 230 from a material more rigid than the material of the valve shaft distal component 140, 240 allows a firmer securement of the user-engagement element 112 with the proximal end 111, 211 of the valve shaft 120, 220 to withstand forces from impact of the circumferential skirt 116 with the radially-inwardly-extending spring support 174 and/or impact of the circumferential neck 118 the radially-inwardly-extending limit shoulder 172, and/or the biasing element 114 with respect to the user-engagement element 112

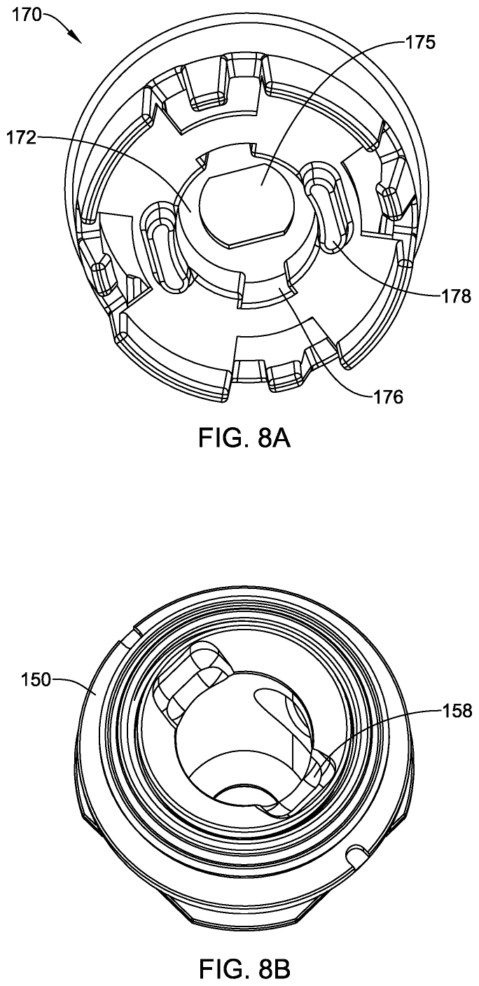

Additionally or alternatively, various features and/or structures may be provided on the valve shaft proximal component 130, 230 to provide hard stops limiting rotational movement of the actuatable member 110 to maintain accurate rotational alignment of the valve-shaft-suction-application port 124, 224 and the valve-well-suction-application port 154. For instance, the valve shaft proximal component 130, 230 may have a non-round cross-sectional shape, and the shaft-receiving through-hole 175 in the cap 170 (through which the valve shaft proximal component 130, 230 extends) may have a corresponding non-round cross-sectional shape such that rotation of the valve shaft 120, 220 with respect to the cap 170 is inhibited, and preferably prevented. In the example of an embodiment illustrated in FIG. 2 and FIG. 3, the valve shaft proximal components 130, 230 may include one or more flats 136, 236, respectively. And, in the example of an embodiment of a cap 170 illustrated in FIG. 8, the shaft-receiving through-hole 175 defined through the radially-inwardly-extending limit shoulder 172 of the cap 170 has a corresponding cross-sectional shape to receive the valve shaft proximal component 130, 230 therethrough rotationally fixing valve shaft proximal component 130, 230, and thus the valve shaft 120, 220, with respect to the cap 170. Moreover, the cap 170 in the illustrated examples of embodiments is configured to be rotationally fixed with respect to the valve well 150. In the example of an embodiment illustrated in FIG. 8A and FIG. 8B, the cap 170 has one or more axially-extending projections 178 engaging corresponding seats 158 in the valve well 150 to fix the cap 170 rotationally with respect to the valve well 150. It will be appreciated that the illustrated engagement features are examples, and other configurations of engagement features between the valve shafts 120, 220, cap 170, and valve well 150 are within the scope and spirit of the present disclosure, the present disclosure not being limited in this regard. Provision of a relatively rigid valve shaft proximal component 130, 230 allows rotational fixing of the valve shaft 120, 220 with respect to the cap 170. As the cap 170 is typically rigid, rotational fixing of the cap 170 with respect to the valve well 150 allows rotational fixing of the valve shaft 120, 220, which is rotationally fixed with respect to the cap 170, to be rotationally fixed to the valve well 150 as well. And, rotational fixing of the valve shaft 120, 220 results in rotational fixing of the valve-shaft-suction-application port 124, 224 with respect to the valve-well-suction-application port 154.

As noted above, in some instances, a suction source may be left on during use of the valve assembly 100 fluidly coupled thereto, with shifting of the actuatable member 110 controlling whether the suction source is in fluid communication with a suction application device coupled with the valve assembly 100. As may be appreciated, it may be desirable to vent or bleed vacuum pressure generated within the valve assembly 100 from the suction source (via the valve-well-suction-source port 152) when the valve assembly 100 is in the off configuration. Rotational fixing of a valve shaft 120, 220 with respect to a cap 170 as in the illustrated examples of embodiments facilitate bleeding or venting of the suction source. For instance, the cap 170 may be provided with bleed passages 176 in fluid communication with the valve shaft suction channel 126, 226, to define a bleed path B for bleeding ambient air to the suction source, such as illustrated in FIG. 8A, FIG. 6A, and FIG. 7A. Ambient air may enter the cap bleed passages 176 via the proximal end 101 of the valve assembly 100, and pass into the valve shaft suction channel 126, 226 via the valve-shaft-suction-application port 124, 224 to bleed the suction source via the valve-shaft-suction-source port 122, 222 and the valve-well-suction-source port 152 when the valve shaft 120, 220 is in the off position illustrated in FIG. 6A and FIG. 7A. Once the valve shaft 120, 220 is in the on position, the valve shaft distal component 140, 240 seals the valve-shaft-suction-application port 124, 224 from the bleed passages 176 to allow suction to be applied to the valve-well-suction-application port 154 without bleeding to outside the valve assembly 100. The seal created by the material of the valve shaft distal component 140 should be capable of cutting off suction to the valve-well-suction-application port 154 when the valve assembly 100 is in an on configuration, as illustrated in FIG. 6B. As illustrated in FIG. 7B, a proximal circumferential seal element 242a on the valve shaft distal component 240 may seal the suction path S from the bleed passages 176 in the cap 170 and ambient air.

Various further benefits of the various aspects, features, components, and structures of a valve shaft and associated seal members, as well as valve assemblies and endoscopes such as described above, in addition to those discussed above, may be appreciated by those of ordinary skill in the art.

It is to be understood by one of ordinary skill in the art that the present discussion is a description of illustrative examples of embodiments only, and is not intended as limiting the broader aspects of the present disclosure. It will be appreciated that principles of the present disclosure may be applied to various medical devices, instruments, tools, etc., such, without limitation, a variety of medical devices, instruments, tools, etc., for accessing anatomical sites and applying suction and/or irrigation thereto, including, for example, endoscopes, gastroscopes, duodenoscopes, catheters, ureteroscopes, bronchoscopes, colonoscopes, arthroscopes, cystoscopes, hysteroscopes, and the like, having integrated features for suction and/or irrigation of anatomical sites. Moreover, principles of the present disclosure may be applied to reusable or single-use devices, instruments, tools, etc.

All apparatuses and methods discussed herein are examples of apparatuses and/or methods implemented in accordance with one or more principles of this disclosure. These examples are not the only way to implement these principles but are merely examples, not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. For instance, various elements and components of a valve assembly described herein may be coupled or engaged directly or indirectly with one another, regardless of how such connections are depicted in the drawings. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated. The various features hereinafter described may be used singly or in any combination thereof. Therefore, the present invention is not limited to only the embodiments specifically described herein, and all substitutes and modifications apparent to those skilled in the art are deemed to be within the spirit, scope, and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A valve shaft configured to shift within the valve well channel of a valve assembly of a medical instrument along an actuation axis and between an off position, in which the valve assembly is in an off configuration, and an on position, in which the valve assembly is in an on configuration, said valve shaft comprising:
a valve shaft proximal component having a proximal end and a distal end and formed of a first material; and
a valve shaft distal component having a proximal end and a distal end and formed of a second material;
wherein:
the first material is more rigid than the second material;
the second material is configured to seal a port defined in the valve well; and
said valve shaft distal component extends distally beyond said valve shaft proximal component.

2. The valve shaft of claim 1, wherein:
said valve shaft distal component is formed of foam; and
said valve shaft proximal component has a distal extension configured to extend into the proximal end of said valve shaft distal component.

3. The valve shaft of claim 2, wherein said distal extension of said valve shaft proximal component comprises one or more barbs engaging within the proximal end of said valve shaft distal component to resist separation of said valve shaft proximal component from said valve shaft distal component.

4. The valve shaft of claim 1, wherein said valve shaft proximal component and said valve shaft distal component are secured together by being at least one of insert molded, overmolded, snap-fitted, interference fitted, welded, bonded, or adhered together.

5. The valve shaft of claim 1, wherein said valve shaft distal component has an outer diameter larger than an inner diameter of the valve well channel of the valve assembly in which said valve shaft is to be extended.

6. The valve shaft of claim 1, wherein:
said valve shaft distal component defines a valve shaft suction channel therethrough extending along the actuation axis and in fluid communication with a valve-well-suction-source port at a distal end of the valve well channel; and
said valve shaft further defines a valve-shaft-suction-application port extending transverse to the actuation axis and in fluid communication with the valve shaft suction channel;
when said valve shaft is in the off position, said valve shaft distal component seals the valve-well-suction-application port from fluid communication with the valve-well-suction-source port; and
when said valve shaft is in the on position, the valve-shaft-suction-application port is in fluid communication with the valve-well-suction-application port to put the valve-well-suction-application port in fluid communication with the valve-well-suction-source port via the valve shaft suction channel.

7. The valve shaft of claim 5, wherein the valve shaft suction channel and the valve shaft suction application port are defined in said valve shaft distal component distal to the distal end of said valve shaft proximal component.

8. The valve shaft of claim 6, wherein said valve shaft distal component includes one or more circumferential seal elements extending circumferentially around and radially outwardly therefrom to seal with respect to the valve well channel.

9. The valve shaft of claim 8, wherein said one or more circumferential seal elements are axially spaced apart from one another along the actuation axis.

10. The valve shaft of claim 9, wherein when said valve shaft is in the off position the valve shaft suction channel is in fluid communication with bleed passages in the valve assembly and with the valve-well-suction-source port, and, when said valve shaft is in the on position, said valve shaft is sealed from fluid communication with the valve shaft suction channel and the valve-well-suction-source port by at least one of said circumferential seal elements.

11. The valve shaft of claim 8, wherein when said valve shaft is in the off position he valve shaft suction channel is in fluid communication with bleed passages in the valve assembly and with the valve-well-suction-source port, and, when said valve shaft is in the on position., said valve shaft is sealed from fluid communication with the valve shaft suction channel and the valve-well-suction-source port by said valve shaft distal component

12. The valve shaft of claim 1, wherein:
said valve shaft is axially shiftable between the off position and on position with respect to a valve cap configured to be coupled to a valve well of the valve assembly; and
said valve shaft proximal component comprises one or more hard stop features engaging the valve cap to limit axial and/or rotational movement of said valve shaft with respect to the valve cap.

13. The valve shaft of claim 12, wherein
the valve cap is configured to be rotationally fixed with respect to the valve well; and
said valve shaft is rotationally fixed with respect to the valve cap and axially shiftable with respect thereto.

14. A valve shaft for a valve assembly configured to be shifted between an off configuration and an on configuration by shifting said valve shaft between an off position and an on position, respectively, said valve shaft comprising:
a valve shaft proximal component formed of a first material; and
a valve shaft distal component formed of a second material;
wherein:
the first material is more rigid than the second material;
the second material is formed of a sealing material capable of sealing a suction path through a valve assembly; and
at least a portion of said valve shaft distal component is formed of only the second material.

15. The valve shaft of claim 14, wherein:
said valve shaft distal component is formed of foam; and
said valve shaft proximal component has a distal extension configured to extend into said valve shaft distal component.

16. The valve shaft of claim 15, wherein said distal extension of said valve shaft proximal component comprises one or more barbs engaging within a proximal end of said valve shaft distal component to resist separation of said valve shaft proximal component from said valve shaft distal component.

17. The valve shaft of claim 14, wherein said valve shaft proximal component and said valve shaft distal component are secured together by being one of insert molded, overmolded, snap-fitted, interference fitted, welded, bonded, or adhered together.

18. A method of forming a valve shaft for valve assembly for a medical instrument, said method comprising:
forming a valve shaft proximal component of a first material; and
forming a valve shaft distal component extending distally away the valve shaft proximal component and of a second material capable of forming a seal with one or more components of the valve assembly, and less rigid than the first material.

19. The method of claim 18, further comprising separately forming and then coupling together the valve shaft proximal component and the valve shaft distal component.

20. The method of claim 18, further comprising molding a proximal end of the valve shaft distal component over a distal end of the valve shaft proximal component and distally beyond the distal end of the valve shaft proximal component.
